(19) United States
(12) Patent Application Publication
(10) Pub. No.: US 2026/0029447 A1
(43) Pub. Date: Jan. 29, 2026

(54) METHODS AND SYSTEMS FOR DEVICE LIFECYCLE PREDICTION

(71) Applicant: Stryker Corporation, Portage, MI (US)

(72) Inventors: Dennis Changmin Sohn, Seattle, WA (US); Jais Jacob, Jacksonville, FL (US); Roger Hildwein, Fall City, WA (US)

(73) Assignee: Stryker Corporation, Portage, MI (US)

(21) Appl. No.: 19/282,942

(22) Filed: Jul. 28, 2025

Related U.S. Application Data
(60) Provisional application No. 63/676,860, filed on Jul. 29, 2024.

(51) Int. Cl. G01R 27/26; A61N 1/362

(52) U.S. Cl. CPC G01R 27/26; A61N 1/3625

(57) ABSTRACT

Methods and systems for determining a lifespan of an accessory device for a medical device such as a defibrillator are provided. An example method includes detecting an impedance of an electrode gel disposed on an electrode, determining environmental conditions to which the accessory device has been exposed, and using the impedance and environmental conditions to predict the expected lifespan of the accessory device.

### CROSS-REFERENCE TO RELATED APPLICATIONS

This application claims priority to and benefit of U.S. Provisional Patent Application No. 63/676,860 filed Jul. 29, 2024, the contents of which are incorporated by reference herein in their entirety.

### BACKGROUND

Medical devices can be used to monitor patients, to administer treatments to patients, or both. Many medical devices include reusable and disposable accessory devices, each of which may have different lifespans and expiry dates. For instance, a defibrillator may be used with disposable accessory devices that include electrode pads. With more complex devices such as a defibrillator, tests can be run, software can be updated, and the devices may be otherwise maintained. With less complex devices including many disposable components, determining the current condition of the device or an accurate expiration date may be more challenging.

### BRIEF DESCRIPTION OF THE FIGURES

The following figures, which form a part of this disclosure, are illustrative of described technology and are not meant to limit the scope of the claims in any manner.

FIG. 1 illustrates an exemplary environment for determining the lifespan of an accessory device.

FIG. 2 illustrates an exemplary environment for determining the lifespan of an accessory device.

FIG. 3 illustrates an exemplary environment for determining the lifespan of an accessory device.

FIGS. 4A-4B illustrate an exemplary medical accessory device.

FIGS. 5A-5B illustrate exemplary packaging for a medical accessory device.

FIGS. 6A-6B illustrate an exemplary process for identifying a lifespan of a medical device accessory.

FIGS. 7A-7B illustrate the use of a predictive model in determining the expected lifespan of a medical accessory device.

FIG. 8 illustrates a change in impedance over time.

FIG. 9 illustrates an example of a medical accessory device connected to an external defibrillator configured to perform various functions described herein.

FIG. 10 illustrates an example environment for training and utilizing a predictive model to determine the expected lifespan of an accessory device.

FIG. 11 illustrates an example system for predicting an expected lifespan of a medical accessory device.

### DETAILED DESCRIPTION

Expiration dates and shelf life of accessory devices can be predicted by assessing the physical and chemical properties of the materials being used, considering the impact of the manufacturing environment on the product's stability, and conducting testing during manufacturing to evaluate the device's performance and functionality. However, once the device leaves the manufacturer, it may be subjected to other conditions that impact the actual lifespan, and which can make the actual lifespan different than the originally predicted lifespan.

Generally, the packaging on an accessory device such as a disposable electrode pad indicates an expected expiration date, as well as a warning to refrain from using the disposable electrode pad if the expiration date has passed. However, relying solely on expected expiration dates to avoid the use of expired medical device accessories has a number of problems, not least of which the expiration date and the actual state of the medical device accessory may not align.

Lifespans and expiration dates are usually determined by manufacturer testing based on a series of assumptions. Such assumptions may or may not hold true in the real world and are generally a conservative estimate of the expected lifespan. Overestimating the expected lifespan may lead to non-working devices, but underestimating a lifespan may lead to premature disposal and waste. For example, an accessory device may be stored under optimal conditions in which the lifespan is longer than predicted. Other accessory devices may be subject to above or below average temperature, moisture, vibration, and atmospheric pressure changes leading to a shorter lifespans. The packaging for the accessory devices may have its own lifespan, and if the packaging fails, the lifespan of the packaged device may be shortened, even though under other conditions, the packaged device would have been expected to have a longer lifespan. For example, the seal of the package may peel or small tears and pinholes may occur during storage or transport.

In their expired state, a medical device and its accessory devices may not be able to perform their intended function which may have serious consequences for patients. For example, if a sufficient amount of water evaporates from a hydrogel coating on an electrode pad, the electrode pad may be unable to accurately detect an electrocardiogram (ECG) of a patient or may be unable to safely or effectively administer an electrical shock (e.g., for the purposes of pacing or defibrillation).

Various implementations described herein address these and other problems by detecting the integrity of a medical accessory device. In some aspects, such detection may take place prior to opening the packaging of the accessory device. In other aspects, detection may take place after opening the packaging of the medical accessory device. Example detection mechanisms as described herein may include sensors that detect the conditions to which the accessory devices have been exposed as well as an electrical characteristic of the accessory device that may be an indication of viability of the device. In various examples, accessory devices can detect and report their readiness statuses prior to being physically coupled to external devices, such as defibrillators or other standalone medical devices.

In some aspects, the systems and methods described herein may determine that the lifespan of the accessory device is within a threshold time period of the lifespan predicted by the manufacturer and will therefore perform as predicted by the manufacturer. In other aspects, the lifespan of the accessory device may be above a threshold time period indicating that it may be used for a time period that exceeds the lifespan predicted by the manufacturer, or below a threshold time period indicating that it may be used for a time period that is less than the lifespan predicted by the manufacturer. Implementations of the present disclosure will now be described with reference to the accompanying figures.

As used herein, the term “accessory device” is a device intended to support, supplement, and/or augment the performance of one or more medical devices. Such accessories include leads, electrode pads, electrical sources, other sensors such as ECG and heart rate sensors, and the like.

As used herein, the terms “standalone medical device,” “medical device,” and their equivalents, can refer to an apparatus that utilizes an accessory device to monitor or to administer a treatment to a patient. In many cases, a standalone medical device is self-powered (e.g., via a battery or a means to connect to mains current), includes input devices (e.g., buttons, touchscreen, etc.) configured to receive user input signals, includes output devices (e.g., speakers, displays, etc.) configured to output signals to users, and includes processing capabilities (e.g., at least one processor) configured to analyze data, report patient conditions, recommend therapy administration, generate a therapy to be administered, or any combination thereof. Examples of standalone medical devices include monitor-defibrillators, automated external defibrillators (AEDs), ventilators, and patient monitors. In various examples, standalone medical devices are reusable.

As used herein, the terms “treatment,” “therapy,” and their equivalents, may refer to a substance, force, or signal that can be administered to the subject for the purpose of resolving a pathology and/or reducing a symptom. Examples of treatments include the administration of one or more electrical shocks (e.g., defibrillation shocks, pace pulses, etc.) to resolve an arrhythmia. Other types of treatments include the administration of assisted ventilation, chest compressions, medications, and the like.

Various implementations of the present disclosure will be described in detail with reference to the drawings, wherein like reference numerals present like parts and assemblies throughout the several views. Additionally, any examples set forth in this specification are not intended to be limiting and merely set forth some of the many possible implementations.

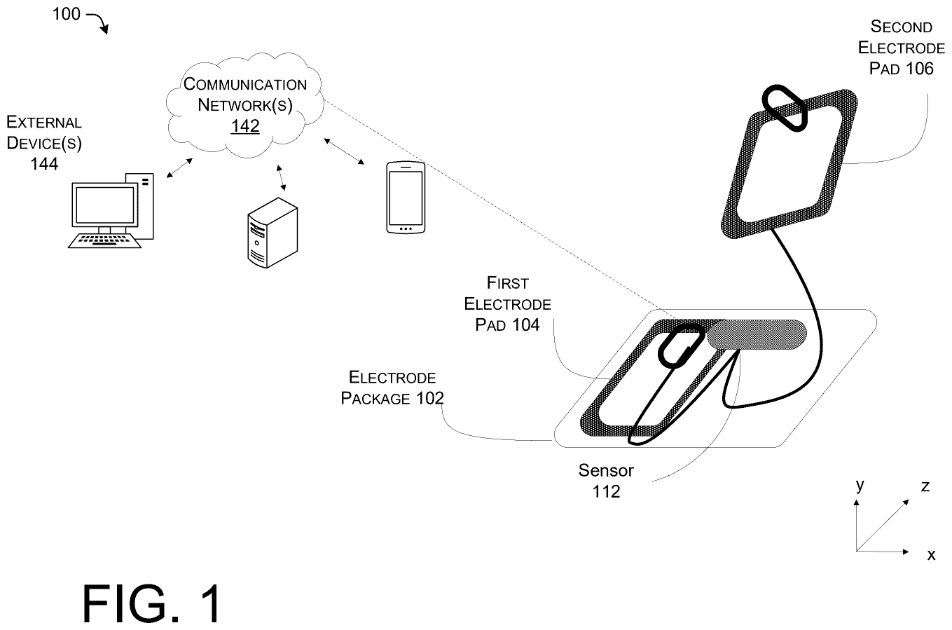

As shown in FIG. 1, an environment 100 may include accessory devices such as first electrode pad 104 and second electrode pad 106. The first electrode pad 104 and the second electrode pad 106 may connect in a wired or wireless fashion to external devices including medical devices. In some aspects, the accessory devices such as the electrode pad 104 may include a transceiver to send and receive data. In additional aspects, the electrode pads such as the first electrode pad 104 and the second electrode pad 106 may include a battery.

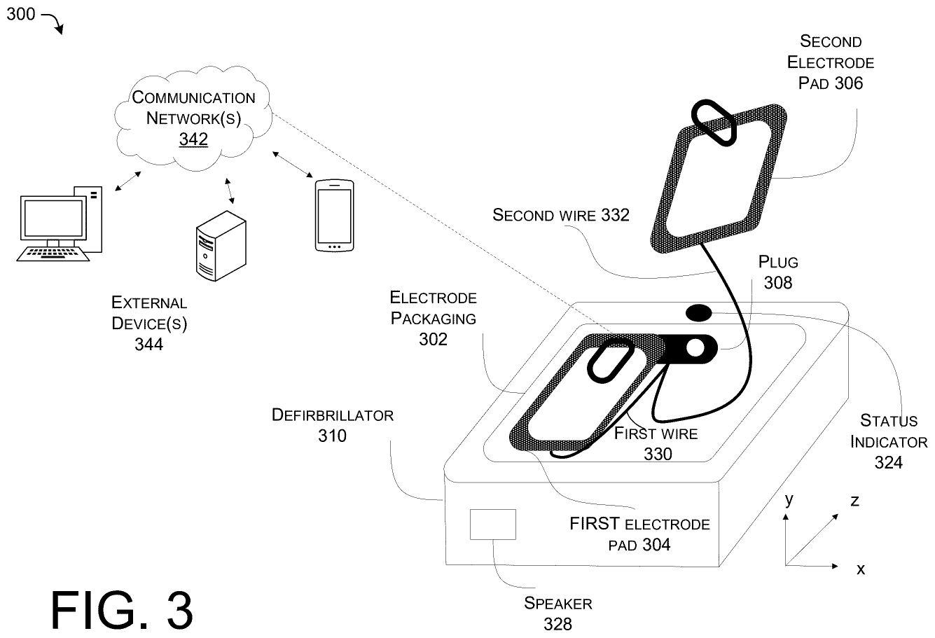

In some aspects, the first electrode pad 104 and the second electrode pad 106, or other components associated with the first electrode pad 104 and the second electrode pad 106 may be in contact with a communication network 142 coupled to external devices such as external device(s) 144. In some aspects, the external device may comprise an application that initiates an automated self-check of the electrodes and/or electrodes connected to the defibrillator shown in further detail in FIG. 3.

Accessory devices such as the first electrode pad 104 and the second electrode pad 106, may be configured to detect an electrical signal from a subject and/or to administer an electrical signal to the subject. In various cases, the first electrode pad 104 and the second electrode pad 106 are configured to be applied externally. For instance, the first electrode pad 104 and the second electrode pad 106 are configured to be disposed on the skin of the subject.

In some aspects, each of the first electrode pad 104 and the second electrode pad 106 further includes a gel including a dielectric material that is configured to enhance the electrical coupling between each electrode and the skin of the subject. The gel, for instance, is a hydrogel. In various cases, the gel includes one or more electrolytes. In some aspects, the gel may be electrically conductive. When the first electrode pad 104 and the second electrode pad 106 are applied to the skin of the subject, the gel is disposed between the skin and the respective electrodes in the first electrode pad 104 and the second electrode pad 106.

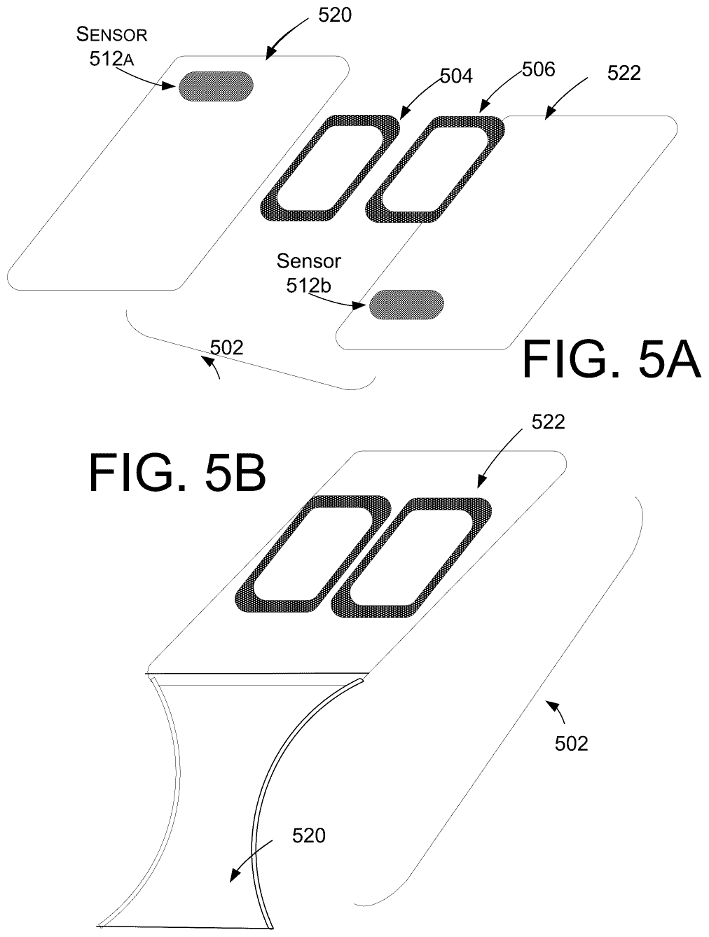

Prior to use, accessory devices such as the first electrode pad 104 and the second electrode pad 106 may be at least partially enclosed in a package to protect and extend the lifespan of the accessory device. Although not specifically illustrated in FIG. 1, the electrode packaging 102 may include a top part (not shown) and a bottom part (shown) as described in further detail in FIGS. 5A and 5B. The packaging may substantially protect the first electrode pad 104 and the second electrode pad 106 from dust, water, and other contaminants. In various cases, electrode packaging 102 may maintain an internal humidity that prevents evaporation of water from the first electrode pad 104 and the second electrode pad 106. For instance, the top half and the bottom half of the package may form a fluid-tight seal prior to removal. According to some cases, a user may open the package by peeling the top layer from the electrode packaging 102 as shown in FIG. 5B. In some cases, the two halves of the electrode packaging 102 are adhered to each other by a fluid-tight adhesive.

The electrode packaging 102 may include one or more sensors such as sensor 112. The sensor may be in contact with or part of the electrode pad(s), the electrode packaging 102, or any combination thereof. In some aspects, the sensor 112 is on the exterior of the packaging as shown in FIG. 5A.

The one or more sensors 112 may be designed to measure a variety of conditions that may impact the lifespan of the accessory device. In some aspects, the sensors are environmental sensors that assess chemical, physical, or biological changes in the electrode pads or the conditions under which the electrode pads are maintained. For example, the one or more sensors 112 may measure humidity, temperature, or motion. While the term sensor is used broadly, the sensor may also be an indicator, tag, or hologram. The sensor may detect chemical, enzymatic, mechanical, electrochemical, or microbiological reactions. In some aspects, the sensor is a diode, resistor, or capacitor. In some aspects, the sensor is a thermocouple. In other aspects, the sensor may be a position sensor, an RFID chip, an accelerometer, piezoelectric crystals, strain gauges, pressure sensor, force sensor, particle sensor, radiation sensor, electrical sensor, colorimetric sensor, thermochromic ink, or gyroscope. The sensor(s) may convey information regarding the conditions to which the accessory has been exposed in a variety of ways. For example, it may change color, shape, size, or may communicate with an exterior communication network such as communication network(s) 142 which may send information to external device(s) 144. The measurements obtained by the one or more sensors may then be used in whole or in part to determine the expected lifespan of the accessory device.

External device(s) 144 may be computing devices including a processor. In some aspects, the processor may be configured to determine the impedance of the electrode gel. While any method of determining impedance may be used, including direct and indirect measurements, in some examples an electrical pulse or current is sent through the system, and resistance is measured (closed loop test). For example, current may be injected using a current supply element in the form of a sine wave signal or DC current. The signal may be boosted through a demodulation technique to amplify the signal to obtain a discernable value reading such as a carrier frequency impedance. In some examples, impedance may be calculated from the resistance. For example, the transfer function, the ratio of a change body impedance (Zb) corresponding voltage signal, may be calculated using the equation Rb/Vb=(Rs+Rb)/Vs (ohms/volt) where Rs=Source resistance, Zb (approx. Rb)=body impedance, Vs=excitation, and Vb=voltage across body resistance. An impedance circuit may use AC or DC current depending on the conditions. In some aspects, the impedance may be calculated using Ohm's law, where R=i/V, where R is the resistance (impedance), “i” is the current, and V is the voltage. For example, there may be an impedance demodulation circuit that generates the DC in-phase and quadrature components of the impedance carrier signal with software-controlled demodulation signals. In other aspects, there may be an impedance circuit that generates the AC in-phase quadrature components from the DC in-phase and quadrature components. In some aspects, an increase in impedance would indicate that the electrical pulse or current would not complete the circuit loop and therefore would be unable to accurately detect physiological parameters from patients, or adequately treat patients.

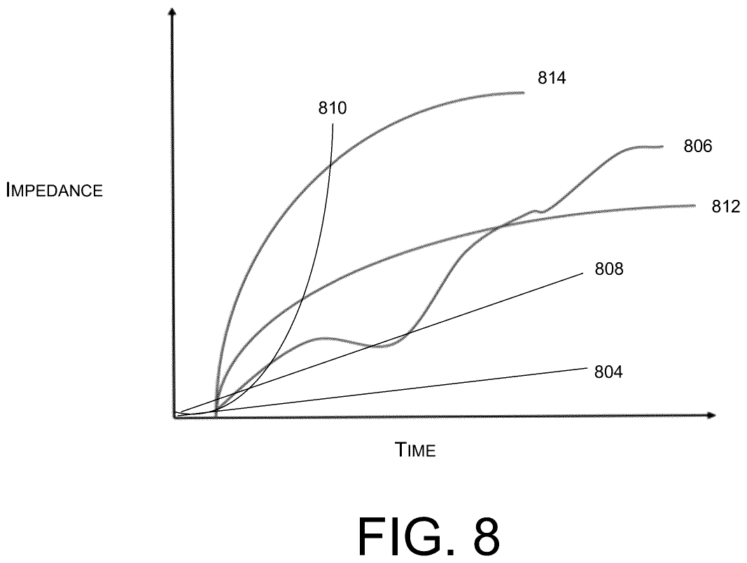

Using the calculated impedance of the gel along with information regarding the environmental conditions that the device has been subject to, the external devices may predict the lifespan of the accessory device. In some aspects, the predicted lifespan may be within a threshold time period. For example, the original manufacture's predicted expiration date may be a threshold time period. In some aspects, the predicted lifespan may be longer than the threshold time period, indicating that the device will last beyond the expected expiration date. In other aspects, the predicted lifespan may be below a threshold time period, indicating the accessory device will expire prior to the expiration date. Generally, the impedance of the electrode gel would be expected to increase over time, however, the pattern and amount of the increase may vary depending on the conditions to which the accessory device has been exposed as shown, for example, in FIG. 8. For example, if the electrode pads are stored under generally optimal conditions in that the temperature, humidity, or vibrations that the accessory device is subject to or the variations of temperature, humidity, or motion are below a threshold amount, the impedance would be expected to increase steadily and slowly as it approaches expiration, resulting in an expiration at or greater than the expected lifespan of the accessory device. In environments where the devices are subject to extreme temperatures or variations in the temperature, humidity, or motion are above a threshold amount, the impedance would appear more random and the general increase in the impedance would be expected to be more rapid, decreasing the lifespan of the electrode pads such as first electrode pad 104 and second electrode pad 106. In some aspects, for example, if the electrodes are cleaned, the impedance could decrease, extending the lifespan of the electrodes.

In some aspects, the resistance of the electrode may be measured using a looping signal (closed loop test) from the defibrillator to the electrode and back to the defibrillator and, using the resistance, impedance may be calculated. For example, the ratio of a change body impedance (Zb) corresponds to a voltage signal using the equation Rb/Vb=(Rs+Rb)/Vs (ohms/volt) where Rs=Source resistance, Zb (approx. Rb)=body impedance, Vs=excitation, and Vb=voltage across body resistance. Such tests may be completed in the electrodes themselves, or after they have been plugged into the defibrillator. For example, the defibrillator may send an electrical current to the electrodes and a signal is returned from the electrodes. Measurements may be a direct measurement of the resistance, or a calculation that derives the resistance measurement.

In some aspects, the determination of lifespan of the device may further include the battery life of the accessory. For example, the charging rate of the accessory device may be determined. As the device ages, the charging rate would be expected to decrease, providing information regarding the lifespan of the accessory device.

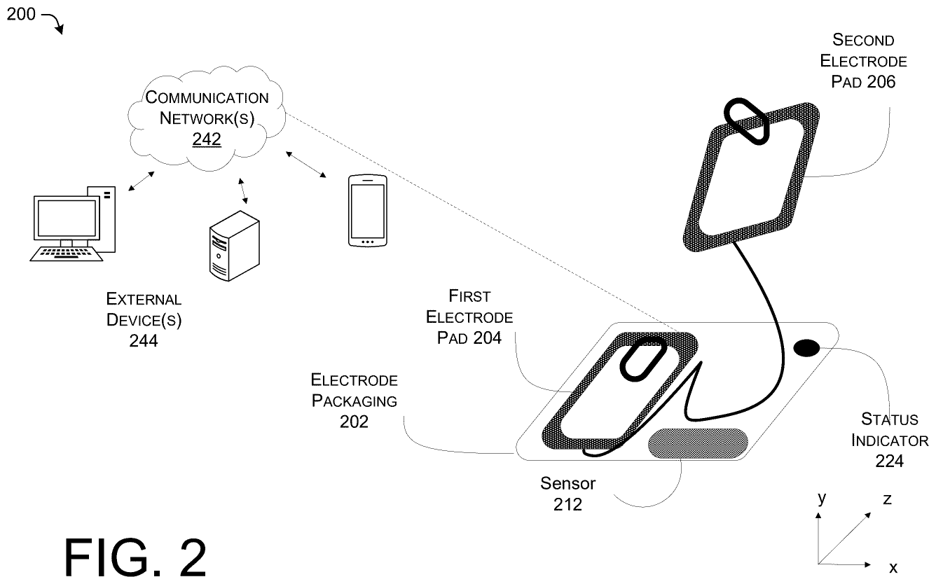

FIG. 2 depicts an environment 200, similar to environment 100, with external device(s) 244 similar to devices 144, a communication network 242 similar to communication network 142, electrode packaging 202 similar to electrode packaging 102, a first electrode pad 204 similar to first electrode pad 104, a second electrode pad 206 similar to second electrode pad 106, and a sensor 212 similar to sensor 112.

In particular examples, information from the sensor may be conveyed by a status indicator such as status indicator 224. Status indicator 224 may be included inside or on the packaging such as electrode packaging 202. In some aspects, the status indicator may indicate that the package has not been stored correctly, for example, it has not been stored at the correct temperature, humidity, or other environmental conditions. In some aspects, it may indicate that the package and therefore the electrode pads have been subjected to an amount of motion over a threshold. The status indicator may provide absolute or nuanced information. For example, it may signal that the accessory device will or will not work as expected or it may reflect the remaining lifespan of the electrode, as, for example, a gradation. The status indicator 224 may be an active or passive indicator. For example, it may change color, shape, size, or may communicate with an exterior communication network such as communication network(s) 242 which may send information to external device(s) 244. In some aspects, the status indicator may be a blinking light that turns on, off, changes color depending on the status of the accessory device, or a pop-up button that indicates an expiration of the accessory device.

FIG. 3 depicts an environment 300 similar to environment 100, with external device(s) 344 similar to devices 144, a communication network 342 similar to communication network 142, electrode package 302 similar to electrode packaging 102, a first electrode pad 304 similar to first electrode pad 104, a second electrode pad 306 similar to second electrode pad 106, and a status indicator 324 similar to status indicator 224.

As shown in FIG. 3, the electrode may be connected to a plug 308. In particular cases, a first wire 330 extends between the electrode of the first electrode pad 304, similar to electrode pad 104, and the plug 308, and a second wire 332 extends between the electrode of the second electrode pad 306, similar to second electrode pad 306 and the plug 308. The plug 308 is an electrical connector configured to removably couple the electrode of the first electrode pad 304 and the electrode of the second electrode pad 306 to a standalone medical device, such as a defibrillator 310. In some aspects all or a portion of the plug 308 may be included in the electrode package 302 for the electrode pads.

In various cases, the defibrillator 310 is configured to monitor and/or administer a therapy to a subject. In particular cases, the defibrillator 310 may be configured to detect one or more physiological parameters (e.g., ECG, transthoracic impedance, etc.) of the subject. The defibrillator 310, in some cases, is configured to analyze the physiological parameter(s) and determine if the subject is exhibiting a shockable heart rhythm (e.g., ventricular fibrillation (VF)). Further, the defibrillator 310 may output a therapy to the subject that treats a shockable heart rhythm. In particular cases, the defibrillator 310 outputs one or more electrical shocks that, when received by the heart of the subject, may cause the heart of the patient to resume a non-shockable heart rhythm, such as a normal sinus rhythm with defined QRS complexes. That is, the defibrillator 310 may be configured to defibrillate the subject.

In the environment 300, sealed electrode package 302 containing the first electrode pad 304 and the second electrode pad 306 is disposed inside of the defibrillator 310. According to some instances, the electrode package 302 may be stored in the defibrillator 310 for an extended period of time prior to use. For instance, the electrode package 302 may be stored for days, weeks, months, or years prior to use. Due to the lengthy storage time, the gel on the first electrode pad 304 and/or the second electrode pad 306 may have dried or otherwise become degraded, which may reduce the efficacy of a defibrillation therapy administered by the defibrillator 310 using the first electrode pad 304 and the second electrode pad 306.

According to various implementations of the present disclosure, a lifespan of the first electrode pad 304 and the second electrode pad 306 can be detected prior to use. In particular cases, the lifespan of the first electrode pad 304 and the second electrode pad 306 can be detected using a sensor. In some aspects, the sensor (not shown) may be disposed inside the plug 308 configured to connect the first electrode pad 304 and the second electrode pad 306 to the defibrillator 310. The lifespan may be indicated visually through, for example status indicator 324, or audibly, for example through speaker 328. In other aspects, a lifespan may be determined by sending a current that loops from the defibrillator to the electrode and back to the defibrillator and measuring the impedance and/or a trend in the impedance. The defibrillator 310 may include a communication module that allows the results to be sent through the communication network 342 to an external device 344. Such a self-check may manually or automatically initiate.

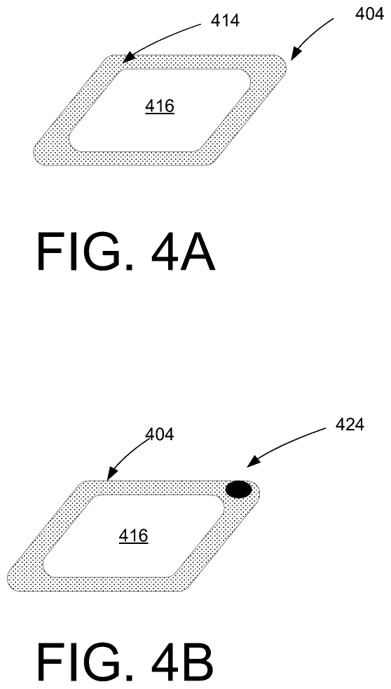

FIGS. 4A-4B depict an electrode pad 404 similar to first electrode pad 104 and second electrode pad 106. Electrode pads may be made of one or more layers including electrode gels, conductive layers, and fabric layers. In some aspects, the electrode may contain an adhesive. The electrode pads are constructed so that at least one electrode gel layer is designed to be in contact with the skin of a patient, though there may be additional electrode gel layers in the electrode. The various layers may be the same or different sizes. In some aspects, an electrode gel may be located in the central portion 416. Some electrode gels or gel layers may be surrounded, for example, with an adhesive layer such as adhesive layer 414 though in other aspects there may be no additional adhesive layer. In some aspects, an electrode pad 404 may include a status indicator such as status indicator 424 as shown in FIG. 4B. The status indicator 424, similar to status indicator 224, may be an active or passive indicator. For example, it may change color, shape, size, or may communicate with an exterior communication network such as communication network(s) 242 which may send information to external device(s) 244. While the status indicator 424 is shown on the underside of electrode pad 404, in some embodiments it may be located on the upper side (that is the non-patient contact side) of an electrode pad.

As shown in FIGS. 5A-5B, the electrode pad 504 similar to first electrode pad 104 and the second electrode pad 506 similar to second electrode pad 106 may be packaged between two layers 520 and 522 forming packaging 502. In some aspects, the two layers may be adhered along an outer edge such that the package is peeled open as shown in FIG. 5B. The packaging 502, similar to electrode packaging 102, may have an interior sensor 512b, an exterior sensor 512a, or both. The sensors 512a and 512b may be designed to measure a variety of conditions that may impact the lifespan of the accessory device including environmental sensors that assess chemical, physical, or biological changes in the electrode pads or the conditions under which the electrode pads are maintained. For example, the one or more sensors 512a and 512b may measure humidity, temperature, or motion. While the term sensor is used broadly, the sensor may also be an indicator, tag, or hologram. The sensor may detect chemical, enzymatic, mechanical, electrochemical, or microbiological reactions. In some aspects, the sensor is a diode. In other aspects, the sensor is a thermocouple. In other aspects, the sensor may be a position sensor, an RFID chip, an accelerometer, piezoelectric crystals, strain gauges, pressure sensor, force sensor, particle sensor, radiation sensors, electrical sensors, colorimetric sensors, thermochromic ink, or gyroscopes. The sensor(s) may convey information regarding the conditions to which the accessory has been exposed in a variety of ways. For example, it may change color, shape, size, or may communicate with an exterior communication network such as communication network(s) 142 which may send information to external device(s) such as external device(s) 144 as shown in FIG. 1.

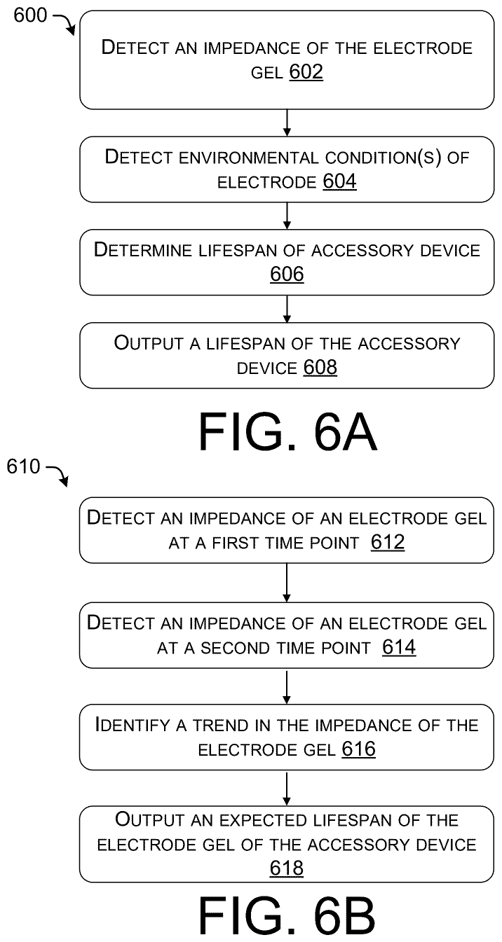

FIGS. 6A and 6B provide processes for determining a lifespan of an accessory device. In process 600 of FIG. 6A, an impedance of the electrode gel is detected at 602. A sensor such as sensor 112 detects the environmental conditions to which the electrode has been exposed at 604. Using the environmental conditions and the impedance, the process 600 determines the lifespan of the accessory device at 606 and the lifespan of the accessory device is then output at 608. The output may be in the form of a status indicator as shown in FIGS. 2-4 or displayed on external devices as shown in FIGS. 1-3.

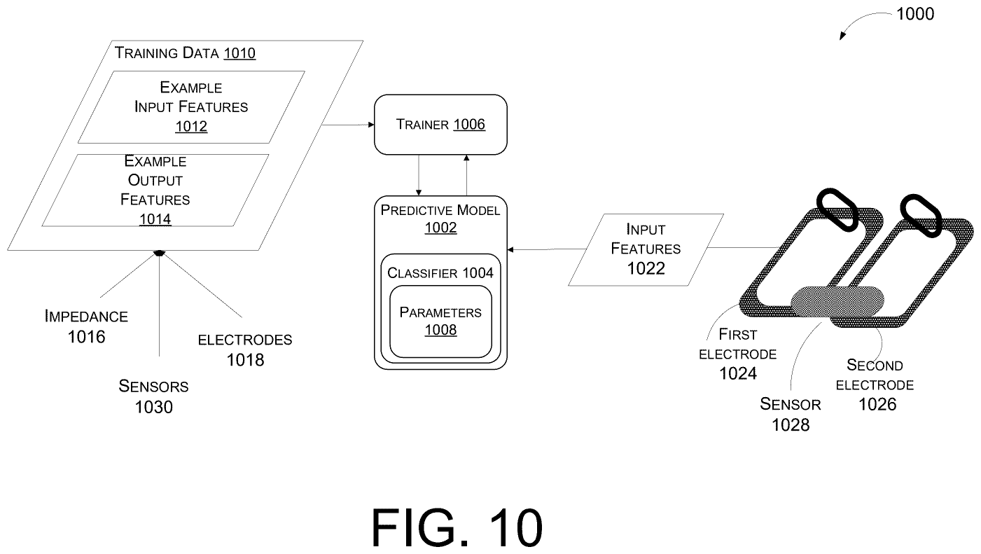

A process 610 is shown at FIG. 6B in which a trend of impedance is determined. Impedance may be determined using any means generally used including direct and/or indirect measurements. In some aspects, impedance may be determined by a closed loop test in which a signal is sent between the defibrillator and the electrode and the impedance is measured. As shown in FIG. 6B, an impedance of an electrode gel is detected at a first time point at 612, using, for example, a closed-loop test. An impedance of an electrode gel is determined at a second time point at 614. A trend between the first time point and the second time point is determined at 616 via a processor and the expected lifespan of the electrode gel of the accessory device is output at 618. The output may be in the form of a status indicator as shown, for example, in FIGS. 2-4 or to external devices, for example, as shown in FIGS. 1-3. In some aspects, the process 610 may include additional measurements, such as changes in the signal-to-noise ratio over time that are greater than threshold changes, changes in resistance, or changes in charge rates. The trend in impedance may be determined from a singular device or a plurality of devices. The plurality of devices may be in the same or different locations and subjected to the same or different environmental conditions or used by the same or different types of providers. In some aspects, the data may be detected globally and analyzed based on one or more types of criteria such as temperature profiles, transport, and handling. In some aspects, the collected data may be used to generate a predictive model for determining the lifespan of the device using, for example, machine learning as described in further detail with reference to FIG. 10.

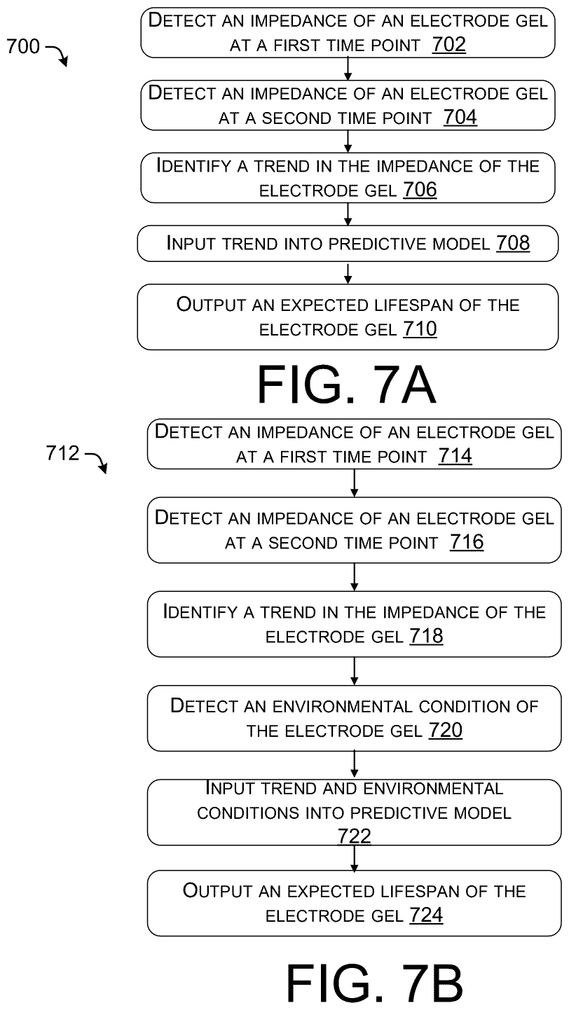

A process 700 as shown in FIG. 7A provides a predictive model for determining an expected lifespan of an electrode gel. As shown in process 700, an impedance of an electrode gel at a first time point is detected at 702. The impedance of the electrode gel at a second time point is detected at 704. The process 700 then identifies a trend in the impedance between the first time point and the second time point at 706. The trend is then put into a predictive model at 708 and the expected lifespan of the electrode gel is determined at 710. The predictive model may be a machine learning model trained to output a lifespan associated with the impedance of an electrode gel on an electrode pad such as first electrode pad 104 and second electrode pad 106 as explained in further detail below. A process 712 as shown in FIG. 7B includes the impact of environmental conditions on the lifespan of the electrode gel of an accessory device. As shown in the process 712, at 714 the impedance of an electrode gel is determined at a first time point. The impedance of an electrode gel is detected at a second time point at 716. The process then determines a trend in the impedance of the electrode gel at 718. The environmental condition of the electrode gel is determined at 720. Detection of an environmental condition may be accomplished, for example, via a sensor such as sensor 112, 212, 512a, and 512b as explained in further detail with reference to FIG. 1, FIG. 2, and FIGS. 5A-5B. The environmental conditions and impedance trend are input into a predictive model at 722. The process 712 determines the lifespan of the accessory device and the lifespan of the accessory device is then output at 724. The output may be in the form of a status indicator as shown in FIGS. 2-4 or to external devices as shown in FIGS. 1-3. In some aspects, the predictive model may also use additional variables such as charge rates, resistance, and signal-to-noise ratio.

The predictive model used, for example in FIGS. 7A and 7B may include machine learning as explained in further detail with reference to FIG. 10. Machine learning algorithms can include regression algorithms (e.g., ordinary least squares regression (OLSR), linear regression, logistic regression, stepwise regression, multivariate adaptive regression splines (MARS), locally estimated scatterplot smoothing (LOESS)), instance-based algorithms (e.g., ridge regression, least absolute shrinkage and selection operator (LASSO), elastic net, least-angle regression (LARS)), decisions tree algorithms (e.g., classification and regression tree (CART), iterative dichotomiser 3 (ID3), Chi-squared automatic interaction detection (CHAID), decision stump, conditional decision trees), Bayesian algorithms (e.g., naïve Bayes, Gaussian naïve Bayes, multinomial naïve Bayes, average one-dependence estimators (AODE), Bayesian belief network (BNN), Bayesian networks), clustering algorithms (e.g., k-means, k-medians, expectation maximization (EM), hierarchical clustering), association rule learning algorithms (e.g., perceptron, back-propagation, hopfield network, Radial Basis Function Network (RBFN)), deep learning algorithms (e.g., Deep Boltzmann Machine (DBM), Deep Belief Networks (DBN), Convolutional Neural Network (CNN), Stacked Auto-Encoders), Dimensionality Reduction Algorithms (e.g., Principal Component Analysis (PCA), Principal Component Regression (PCR), Partial Least Squares Regression (PLSR), Sammon Mapping, Multidimensional Scaling (MDS), Projection Pursuit, Linear Discriminant Analysis (LDA), Mixture Discriminant Analysis (MDA), Quadratic Discriminant Analysis (QDA), Flexible Discriminant Analysis (FDA)), Ensemble Algorithms (e.g., Boosting, Bootstrapped Aggregation (Bagging), AdaBoost, Stacked Generalization (blending), Gradient Boosting Machines (GBM), Gradient Boosted Regression Trees (GBRT), Random Forest), SVM (support vector machine), supervised learning, unsupervised learning, semi-supervised learning, etc. Additional examples of architectures include neural networks such as ResNet-50, ResNet-101, VGG, DenseNet, PointNet, and the like. In some examples, the ML model discussed herein may comprise PointPillars, SECOND, top-down feature layers (e.g., see U.S. patent application Ser. No. 15/963,833, which is incorporated in its entirety herein), and/or VoxelNet. Architecture latency optimizations may include MobilenetV2, Shufflenet, Channelnet, Peleenet, and/or the like. The ML model may comprise a residual block such as Pixor, in some examples.

In some examples, external devices such as, for example, external device(s) 144 and similar devices shown in FIGS. 2 and 3 may determine the lifespan of the first electrode pad 104 and the second electrode pad 106 using one or more of data from the sensors, analysis of the impedance of the electrode gel on the electrode pads, analysis of the resistance of the electrode gel on the electrode pads, analysis of the signal-to-noise ratio, analysis of battery life and/or analysis of the geographic location. For example, the analysis may include some or all devices in a specific geographic location or some or all devices used by a particular type of provider, or a combination thereof. The analysis of the data may include acquiring measurements at one or more points in time and determining trends in the various measurements over periods of time. In some aspects, the analysis of the data may include average readings for a plurality of devices in a specific area or stored under a specific set of circumstances (for example, in a vehicle). In some aspects, the analysis of data may include other factors related to a geographic location such as temperature, elevation, humidity, and the like.

After the lifespan of the electrode pad is determined, one or more display settings of an output device may be adjusted to reflect the lifespan of the device. In an example, the display settings include changing a brightness, contrast, and/or a color of a portion of the display on the external device(s) 144 such as the portion of the display showing the expected lifespan. In some aspects, the lifespan may be displayed by a status indicator. The lifespan, in some examples, relates to an output from a machine learning model.

FIG. 8 is a graph illustrating predicted changes in impedance over time. Under ideal conditions, that is with variation of environmental conditions below a threshold during the storage of the accessory device, the impedance of the electrode gel would be predicted by the machine learning model to increase slowly over time as shown at 804. Under conditions in which the electrodes are stored in such a way that there is rapid degradation at the connection points, the impedance may increase logarithmically as shown at 814. In other aspects, the rapid degradation at the connection points may create a linear increase in impedance as shown at 808. For example, if the accessory device was located in a climate that was hot and dry, the impedance would be expected to increase more rapidly as shown at 814 or 808 than if the accessory device was in a climate that was more temperate. Similarly, if the accessory device was located in a climate that reached freezing temperatures for sustained periods of time, the impedance would also be expected to increase more rapidly than if the accessory device was in a climate that was more temperate. Under conditions in which there was a lot of variability in environmental conditions, the impedance would be expected to have increased variability and the overall increase in impedance would occur more quickly than under ideal conditions as shown at 806. In some aspects, the line shown at 806 may indicate that the contacts are periodically cleaned, improving the impedance. This may include any type of cleaning, for example, insertion/removal cycling, physical removal, high voltage shock, or chemical removal. In some instances, the increase in impedance may be exponential as shown at 810, for example when there is a rapid decline in the health and performance of the accessory device.

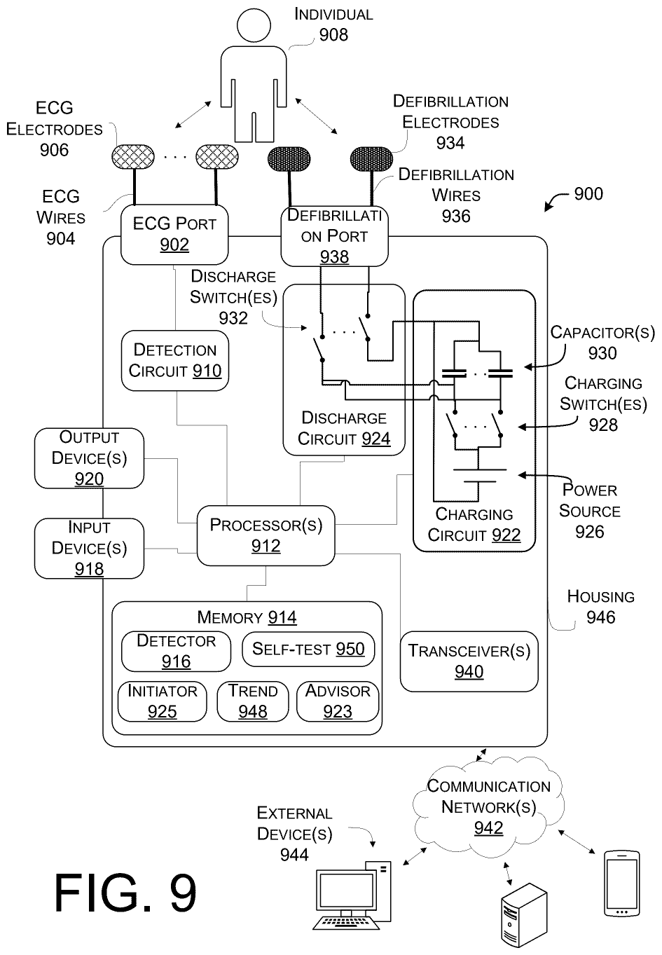

FIG. 9 illustrates an example of an external defibrillator 900 configured to perform various functions described herein using the accessory devices described herein. For example, the external defibrillator 900 is the defibrillator 310 described above with reference to FIG. 3.

The external defibrillator 900 includes an electrocardiogram (ECG) port 902 connected to multiple ECG wires 904. In some cases, the ECG wires 904 are removeable from the ECG port 902. For instance, the ECG wires 904 are plugged into the ECG port 902. The ECG wires 904 are connected to ECG electrodes 906, respectively. The ECG electrodes 906 are similar to electrode pads 104, 106, 204, 206, 304, 306, 404, 504, and 506 as described with reference to FIGS. 1-5B. In various implementations, the ECG electrodes 906 are disposed on different locations on an individual 908. A detection circuit 910 is configured to detect relative voltages between the ECG electrodes 906. These voltages are indicative of the electrical activity of the heart of the individual 908.

In various implementations, the ECG electrodes 906 are in contact with the different locations on the skin of the individual 908. In some examples, a first one of the ECG electrodes 906 is placed on the skin between the heart and right arm of the individual 908, a second one of the ECG electrodes 906 is placed on the skin between the heart and left arm of the individual 908, and a third one of the ECG electrodes 906 is placed on the skin between the heart and a leg (either the left leg or the right leg) of the individual 908. In these examples, the detection circuit 910 is configured to measure the relative voltages between the first, second, and third ECG electrodes 906. Respective pairings of the ECG electrodes 906 are referred to as “leads,” and the voltages between the pairs of ECG electrodes 906 are known as “lead voltages.” In some examples, more than three ECG electrodes 906 are included, such that 5-lead or 12-lead ECG signals are detected by the detection circuit 910.

The detection circuit 910 includes at least one analog circuit, at least one digital circuit, or a combination thereof. The detection circuit 910 receives the analog electrical signals from the ECG electrodes 906, via the ECG port 902 and the ECG wires 904. In some cases, the detection circuit 910 includes one or more analog filters configured to filter noise and/or artifact from the electrical signals. The detection circuit 910 includes an analog-to-digital converter (ADC) in various examples. The detection circuit 910 generates a digital signal indicative of the analog electrical signals from the ECG electrodes 906. This digital signal can be referred to as an “ECG signal” or an “ECG.”

In some cases, the detection circuit 910 further detects an electrical impedance between at least one pair of the ECG electrodes 906. For example, the detection circuit 910 includes, or otherwise controls, a power source that applies a known voltage (or current) across a pair of the ECG electrodes 906 and detects a resultant current (or voltage) between the pair of the ECG electrodes 906. The impedance is generated based on the applied signal (voltage or current) and the resultant signal (current or voltage). In various examples, the detection circuit 910 includes one or more analog filters configured to filter noise and/or artifact from the resultant signal. The detection circuit 910 generates a digital signal indicative of the impedance using an ADC. This digital signal can be referred to as an “impedance signal” or an “impedance.” In some aspects, the impedance may be the impedance of the electrodes. In other aspects, the impedance may be used to determine if the individual 908 is exhibiting a shockable rhythm.

The detection circuit 910 provides the ECG signal and/or the impedance signal to one or more processors 912 in the external defibrillator 900. In some implementations, the processor(s) 912 includes a central processing unit (CPU), a graphics processing unit (GPU), digital signal processing unit (DPU), other processing unit or component known in the art, or any combination thereof.

The processor(s) 912 is operably connected to memory 914. In various implementations, the memory 914 is volatile (such as random access memory (RAM)), non-volatile (such as read only memory (ROM), flash memory, etc.) or some combination of the two. The memory 914 stores instructions that, when executed by the processor(s) 912, causes the processor(s) 912 to perform various operations. In various examples, the memory 914 stores methods, threads, processes, applications, objects, modules, any other sort of executable instruction, or a combination thereof. In some cases, the memory 914 stores files, databases, or a combination thereof. In some examples, the memory 914 includes, but is not limited to, RAM, ROM, electrically erasable programmable read-only memory (EEPROM), flash memory, or any other memory technology. In some examples, the memory 914 includes one or more of CD-ROMs, digital versatile discs (DVDs), content-addressable memory (CAM), or other optical storage, magnetic cassettes, magnetic tape, magnetic disk storage or other magnetic storage devices, or any other medium which can be used to store the desired information and which can be accessed by the processor(s) 912 and/or the external defibrillator 900. In some cases, the memory 914 at least temporarily stores the ECG signal and/or the impedance signal.

In various examples, the memory 914 includes a detector 916, which causes the processor(s) 912 to determine, based on the ECG signal and/or the impedance signal, whether the individual 908 is exhibiting a particular heart rhythm. For instance, the processor(s) 912 determines whether the individual 908 is experiencing a shockable rhythm that is treatable by defibrillation. Examples of shockable rhythms include ventricular fibrillation (VF) and ventricular tachycardia (VT). In some examples, the processor(s) 912 determines whether any of a variety of different rhythms (e.g., asystole, sinus rhythm, atrial fibrillation (AF), etc.) are present in the ECG signal.

The processor(s) 912 is operably connected to one or more input devices 918 and one or more output devices 920. Collectively, the input device(s) 918 and the output device(s) 920 function as an interface between a user and the defibrillator 900. The input device(s) 918 is configured to receive an input from a user and includes at least one of a switch, a keypad, a cursor control, a touch-sensitive display, a voice input device (e.g., a microphone), a haptic feedback device (e.g., a gyroscope), or any combination thereof. The output device(s) 920 includes at least one of a display, a speaker, a haptic output device, a printer, a light indicator such as an LED, or any combination thereof. In various examples, the processor(s) 912 causes a display among the input device(s) 918 to visually output a waveform of the ECG signal and/or the impedance signal. In some implementations, the input device(s) 918 includes one or more touch sensors, the output device(s) 920 includes a display screen, and the touch sensor(s) are integrated with the display screen. Thus, in some cases, the external defibrillator 900 includes a touchscreen configured to receive user input signal(s) and visually output physiological parameters, such as the ECG signal and/or the impedance signal.

In some examples, the memory 914 includes an advisor 923, which, when executed by the processor(s) 912, causes the processor(s) 912 to generate advice and/or control the output device(s) 920 to output the advice to a user (e.g., a rescuer). In some examples, the processor(s) 912 provides, or causes the output device(s) 920 to provide, an instruction to perform CPR on the individual 908. In some cases, the processor(s) 912 evaluates, based on the ECG signal, the impedance signal, or other physiological parameters, CPR being performed on the individual 908 and causes the output device(s) 920 to provide feedback about the CPR in the instruction. According to some examples, the processor(s) 912, upon identifying that a shockable rhythm is present in the ECG signal, causes the output device(s) 920 to output an instruction and/or recommendation to administer a defibrillation shock to the individual 908.

The memory 914 also includes an initiator 925 which, when executed by the processor(s) 912, causes the processor(s) 912 to control other elements of the external defibrillator 900 in order to administer a defibrillation shock to the individual 908. In some examples, the processor(s) 912 executing the discharge circuit 924 selectively causes the administration of the defibrillation shock based on determining that the individual 908 is exhibiting the shockable rhythm and/or based on an input from a user (received, e.g., by the input device(s) 918. In some cases, the processor(s) 912 causes the defibrillation shock to be output at a particular time, which is determined by the processor(s) 912 based on the ECG signal and/or the impedance signal.

The memory 914 also includes a trend 948 which receives sensor measurements and impedance measurements to determine the impedance over time. Measurements may be received from a plurality of time points and a model may be applied that predicts the expiration of the accessory device as shown, for example, in FIG. 7B, though other processes may also be used.

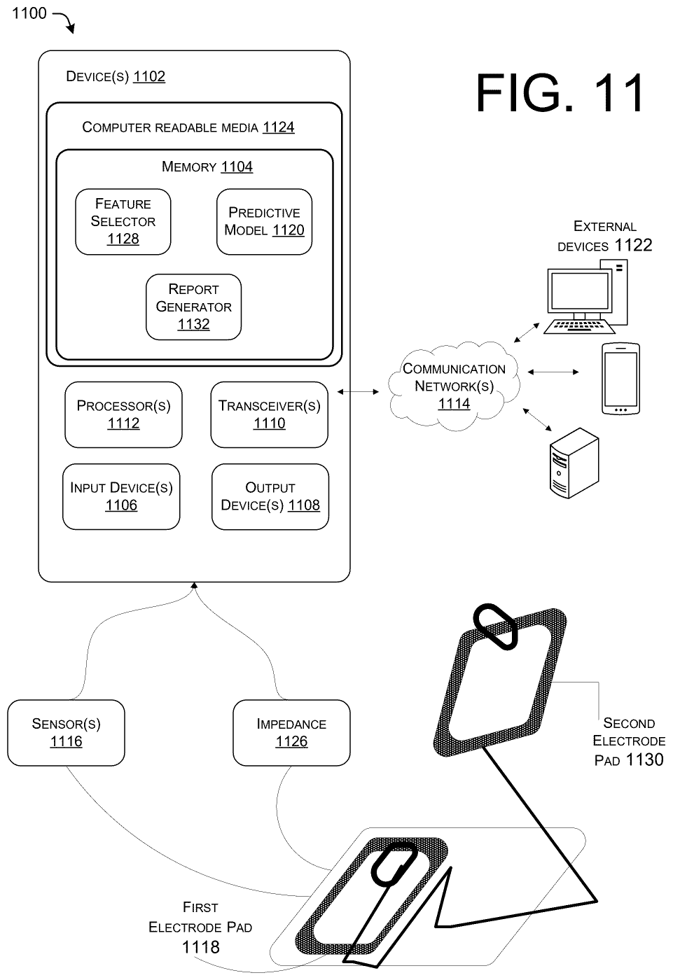

The memory 914 also includes a self-test 950. The self-test may be configured to cause the device to send a test signal to the electrode to determine the impedance of the electrode gel of the accessory device, using, for example, a closed-loop test. The impedance measurement may be used, for example in the trend 948 to determine the expected lifespan of the accessory device. In some aspects, the self-test may be executed by the electrodes such that the electrodes send and receive a signal and use the signal to determine the expected lifespan of the electrode(s). The results of the test signal may be analyzed by a processor such as processor(s) 912 or by a similar type of processor in the electrode. In some aspects, the results of the self-test are sent to external devices such as external device(s) 144 in FIG. 1 or the similar devices in FIGS. 2 and 3. In other aspects, the self-test may be executed by a second device such as device 1102 in FIG. 11.

The processor(s) 912 is operably connected to a charging circuit 922 and a discharge circuit 924. In various implementations, the charging circuit 922 includes a power source 926, one or more charging switches 928, and one or more capacitors 930. The power source 926 includes, for instance, a battery. The processor(s) 912 initiates a defibrillation shock by causing the power source 926 to charge at least one capacitor among the capacitor(s) 930. For example, the processor(s) 912 activates at least one of the charging switch(es) 928 in the charging circuit 922 to complete a first circuit connecting the power source 926 and the capacitor to be charged. Then, the processor(s) 912 causes the discharge circuit 924 to discharge energy stored in the charged capacitor across a pair of defibrillation electrodes 934, which are in contact with the individual 908. For example, the processor(s) 912 deactivates the charging switch(es) 928 completing the first circuit between the capacitor(s) 930 and the power source 926 and activates one or more discharge switches 932 completing a second circuit connecting the charged capacitor 930 and at least a portion of the individual 908 disposed between defibrillation electrodes 934.

The energy is discharged from the defibrillation electrodes 934 in the form of a defibrillation shock. For example, the defibrillation electrodes 934 are connected to the skin of the individual 908 and located at positions on different sides of the heart of the individual 908, such that the defibrillation shock is applied across the heart of the individual 908. The defibrillation shock, in various examples, depolarizes a significant number of heart cells in a short amount of time. The defibrillation shock, for example, interrupts the propagation of the shockable rhythm (e.g., VF or VT) through the heart. In some examples, the defibrillation shock is 200 J or greater with a duration of about 0.015 seconds. In some cases, the defibrillation shock has a multiphasic (e.g., biphasic) waveform. The discharge switch(es) 932 are controlled by the processor(s) 912, for example. In various implementations, the defibrillation electrodes 934 are connected to defibrillation wires 936. The defibrillation wires 936 are connected to a defibrillation port 938, in implementations. According to various examples, the defibrillation wires 936 are removable from the defibrillation port 938. For example, the defibrillation wires 936 are plugged into the defibrillation port 938.

In various implementations, the processor(s) 912 is operably connected to one or more transceivers 940 that transmit and/or receive data over one or more communication networks 942. For example, the transceiver(s) 940 includes a network interface card (NIC), a network adapter, a local area network (LAN) adapter, or a physical, virtual, or logical address to connect to the various external devices and/or systems. In various examples, the transceiver(s) 940 includes any sort of wireless transceivers capable of engaging in wireless communication (e.g., radio frequency (RF) communication). For example, the communication network(s) 942 includes one or more wireless networks that include a 3^{rd} Generation Partnership Project (3GPP) network, such as a Long Term Evolution (LTE) radio access network (RAN) (e.g., over one or more LTE bands), a New Radio (NR) RAN (e.g., over one or more NR bands), or a combination thereof. In some cases, the transceiver(s) 940 includes other wireless modems, such as a modem for engaging in WI-FI®, WIGIG®, WIMAX®, BLUETOOTH®, or infrared communication over the communication network(s) 942.

The defibrillator 900 is configured to transmit and/or receive data (e.g., ECG data, impedance data, data indicative of one or more detected heart rhythms of the individual 908, data indicative of one or more defibrillation shocks administered to the individual 908, etc.) with one or more external device(s) 944 via the communication network(s) 942. The external device(s) 944 include, for instance, mobile devices (e.g., mobile phones, smart watches, etc.), Internet of Things (IoT) devices, computers (e.g., laptop devices, servers, etc.), or any other type of computing device configured to communicate over the communication network(s) 942. In some examples, the external device(s) 944 is located remotely from the defibrillator 900, such as at a remote clinical environment (e.g., a hospital). According to various implementations, the processor(s) 912 causes the transceiver(s) 940 to transmit data to the external device(s) 944. In some cases, the transceiver(s) 940 receives data from the external device(s) 944 and the transceiver(s) 940 provide the received data to the processor(s) 912 for further analysis.

In various implementations, the external defibrillator 900 also includes a housing 946 that at least partially encloses other elements of the external defibrillator 900. For example, the housing 946 encloses the detection circuit 910, the processor(s) 912, the memory 914, the charging circuit 922, the transceiver(s) 940, or any combination thereof. In some cases, the input device(s) 918 and output device(s) 920 extend from an interior space at least partially surrounded by the housing 946 through a wall of the housing 946. In various examples, the housing 946 acts as a barrier to moisture, electrical interference, and/or dust, thereby protecting various components in the external defibrillator 900 from damage.

In some implementations, the external defibrillator 900 is an automated external defibrillator (AED) operated by an untrained user (e.g., a bystander, layperson, etc.) and can be operated in an automatic mode. In automatic mode, the processor(s) 912 automatically identifies a rhythm in the ECG signal, makes a decision whether to administer a defibrillation shock, charges the capacitor(s) 930, discharges the capacitor(s) 930, or any combination thereof. In some cases, the processor(s) 912 controls the output device(s) 920 to output (e.g., display) a simplified user interface to the untrained user. For example, the processor(s) 912 refrains from causing the output device(s) 920 to display a waveform of the ECG signal and/or the impedance signal to the untrained user, in order to simplify operation of the external defibrillator 900.

In some examples, the external defibrillator 900 is a monitor-defibrillator utilized by a trained user (e.g., a clinician, an emergency responder, etc.) and can be operated in a manual mode or the automatic mode. When the external defibrillator 900 operates in manual mode, the processor(s) 912 cause the output device(s) 920 to display a variety of information that may be relevant to the trained user, such as waveforms indicating the ECG data and/or impedance data, notifications about detected heart rhythms, and the like.

FIG. 10 illustrates an example environment 1000 for training and utilizing a predictive model 1002 to determine the expected lifespan of an accessory device. The predictive model 1002, for instance, is the predictive model for expected lifespan of an accessory device such as the accessory device described above with reference to FIGS. 1-5B. In various implementations, the predictive model 1002 includes a classifier 1004, which may include one or more machine learning (ML) models. A trainer 1006, for instance, is configured to optimize various parameters 1008 of the classifier 1004 based on training data 1010.

The training data 1010 includes example expected lifespan of an accessory device 1012 and example categories 1014. The example expected lifespan of an accessory device 1012, in various cases, is obtained using electrodes 1018 subject to a variety of conditions by the manufacturer as well as data collected in the field, for example data collected by sensors 1030. The examples may additionally include measurements of impedance 1016 over time. The example categories 1014 may include categorizations of chemical, biochemical, or physical changes to the electrodes 1018 as well as changes to the impedance 1016.

The example categories 1014 may include categorizations of various conditions to which the accessory device may be exposed including temperature, humidity, atmospheric pressure, and vibration among others. For example, the example categories 1014 may be generated based on expected lifespan of an accessory device subjected to a variety of conditions. The example categories 1014 may additionally or solely include impedance values of electrodes subject to a variety of conditions and the impact of those conditions on the impedance and/or lifespan of electrodes and electrode gel.

The classifier 1004 includes one or more model types. For instance, the classifier 1004 may include an artificial neural network. An artificial neural network includes various layers that respectively process input data. For example, an artificial neural network includes an input layer, one or more hidden layers, and an output layer. The input layer performs a pre-processing operation on the input data. The hidden layer(s) may perform various processing operations on the output from the input layer. The output layer, in various cases, processes the output from the hidden layer(s). Each layer, in some cases, includes one or more nodes, which are defined by individual operations. In various cases, the hidden layer(s) include nodes that are connected to each other in parallel and/or series. Examples of artificial neural networks include feedforward neural networks, multi-layer perceptrons (MLPs), convolutional neural networks (CNNs), and backpropagation models. In various implementations, the operations performed by the layers and/or nodes within an artificial neural network included in the classifier 1004 is defined according to the parameters 1008. For example, the parameters 1008 may include weights, thresholds, filters, kernels, or other data objects that are utilized to perform operations of the classifier 1004.

In some implementations, the classifier 1004 includes a nearest-neighbor model. One example of a nearest-neighbor model includes a k-nearest neighbor model. For example, a nearest-neighbor model defines various “neighbors,” which are points within a feature space, with associated class labels. When a new data point is mapped to the feature space, the new data point is classified based on the proximity (e.g., Euclidian distance, Manhattan distance, Minkowski distance, etc.) of its “neighbors” to the new data point as well as their associated classes. In some cases, the new data point is classified as belonging to a particular class if greater than a threshold number of neighbors within a threshold distance of the new data point are members of the class. For instance, the parameters 1008 may include k (e.g., the number of neighbors compared to the new data point), the threshold distance, and so on.

In various cases, the classifier 1004 includes a regression analysis model. The regression analysis model, for example, is defined by a regression function that defines relationships between one or more independent variables and one or more dependent variables. The regression function may further define one or more unknown parameters that define a relationship between the independent and dependent variables. In various implementations, the unknown parameters and/or the type of regression function (e.g., linear, quadratic, etc.), is defined according to the parameters 1008.

In some cases, the classifier 1004 includes a clustering model. In various cases, a clustering model maps various data points (e.g., training data) to a feature space. Based on the proximity of groups of those data points in the features pace, one or more “clusters” are defined. An additional data point may be classified according to one or more of the clusters based on its proximity to the clusters (e.g., a center of the clusters, a boundary of the cluster, etc.). Examples of clustering models include k-means clustering, mean-shift clustering, expectation-maximization (EM) clustering, and agglomerative hierarchical clustering. The parameter(s) 1008, for example, include a threshold proximity within which a new data point is classified within a cluster, a density of points used to define a cluster, and the like.

In various examples, the classifier 1004 includes a principal component analysis model. In various implementations, a principal component analysis defines a collection of principal components of unit vectors within a coordinate space based on a data set (e.g., training data). The model, for example, is an orthogonal linear transformation of the data set. Various weights of the model, for example, are included in the parameter(s) 1008.

The classifier 1004, in some implementations, includes a gradient boosting model. For example, the gradient boosting model is defined as a collection of prediction models (e.g., decision trees) that iteratively classify observed data. In various cases, the type of prediction model, weights in the prediction models, and the like, are defined by the parameter(s) 1008.

The classifier 1004, for example, includes a random forest. The random forest, for instance, includes multiple decision trees that classify data in an ensemble fashion. In various implementations, the decision trees are defined by the parameter(s) 1008.

In various implementations of the present disclosure, the trainer 1006 is configured to optimize the parameters 1008 based on the training data 1010. For example, the trainer 1006 may input first example features among the example expected lifespan of an accessory device 1012 into the predictive model 1002, and may receive a predicted category. The trainer 1006 may compute a loss (e.g., determine a discrepancy) between a first example category (corresponding to the first electrode) among the example categories 1014 and the predicted category. Further, the trainer 1006 may alter the parameters 1008 in order to minimize the loss. In various cases, the trainer 1006 optimizes the parameters 1008 iteratively based on the entire set of the training data 1010.

In various implementations, the optimization of the parameters 1008 enables the predictive model 1002 to identify predictive attributes of the example expected lifespan of an accessory device including first electrode 1024 and second electrode 1026 that are correlated to or otherwise associated with the example output features 1014. The predictive model 1002 may therefore classify expected lifespan based on the variety of conditions the accessory device has been subject to by recognizing or otherwise identifying the predictive attributes.

Once the parameters 1008 are optimized, the predictive model 1002 may be ready to classify a new set of data. For example, the predictive model 1002 may receive input data including features 1022 of first electrode 1024, second electrode 1026, and sensors 1028. The features 1022, for instance, may include one or more of the predictive attributes. The predictive model 1002 may perform various operations on the input data based on the trained classifier 1004 and the optimized parameters 1008.

Although FIG. 10 is primarily described as referring to supervised learning, implementations are not so limited. In various cases, the training data 1010 omits the example categories 1014 and the trainer 1006 is configured to optimize the parameters 1008 using the example expected lifespan of an accessory device 1012 and an unsupervised learning technique.

FIG. 11 illustrates a system 1100 configured to perform various functions described herein. For example, the system 1100 may be configured to execute the processes of FIGS. 7A and 7B using machine learning.

In various implementations, the operation of the device 1102 may be controlled by at least one processor 1112. The processor(s) is configured to analyze the signals from the sensor(s) 1116 measuring the environment of the first electrode pad 1118 and the second electrode pad 1130. In some aspects, the processor(s) may be configured to analyze the impedance of a gel of the first electrode pad 1118 and the second electrode pad 1130. The impedance may be determined, for example, using a closed loop test in which a signal is sent through the electrode. For example, current may be injected using a current supply element. The injected current may be in the form of a sine wave signal or DC current. In some aspects, the signal may be boosted through a demodulation technique to amplify the signal for discernable value reading such as a carrier frequency impedance. Using the resistance, impedance may be calculated. In some aspects, the ratio of a change body impedance (Zb) corresponding voltage signal may be calculated using the equation Rb/Vb=(Rs+Rb)/Vs (ohms/volt) where Rs=Source resistance, Zb (approx. Rb)=body impedance, Vs=excitation, and Vb=voltage across body resistance. An impedance circuit may use AC or DC current depending on the conditions. In some aspects, the impedance may be calculated using Ohm's law, where R=i/V, where R is the resistance (impedance), “i” is the current, and V is the voltage.

In various implementations, the device 1102 includes at least one transceiver 1110 configured to communicate with at least one external device 1122 over one or more communication networks 1114. Any communication network described herein can be included in the communication network(s) 1114 illustrated in FIG. 11. The external device(s) 1122, for example, includes at least one of a monitor, a mobile phone, a server, or a computing device. In some implementations, the transceiver(s) 1110 is configured to communicate with the external device(s) 1122 by transmitting and/or receiving signals in a wired fashion and/or wirelessly. For example, the transceiver(s) 1110 includes a NIC, a network adapter, a LAN adapter, or a physical, virtual, or logical address to connect to the various external devices and/or systems. In various examples, the transceiver(s) 1110 includes any sort of wireless transceivers capable of engaging in wireless communication (e.g., RF communication). For example, the communication network(s) 1114 includes one or more wireless networks that include a 3GPP network, such as an LTE RAN (e.g., over one or more LTE bands), an NR RAN (e.g., over one or more NR bands), or a combination thereof. In some cases, the transceiver(s) 1110 includes other wireless modems, such as a modem for engaging in WI-FI®, WIGIG®, WIMAX®, BLUETOOTH®, or infrared communication over the communication network(s) 1114. The signals, in various cases, encode data in the form of data packets, datagrams, or the like.

In various cases, the processor(s) 1112 generates a lifespan based on data encoded in the signals received from the sensors 1116 and impedance measurement 1126. For instance, the signals include impedance measurements and/or environmental conditions and the like, and the processor(s) 1112 outputs a lifespan that can be displayed on one or more of the external devices 1122.

According to some examples, the input device(s) 1106 receives information from one or more sensors 1116. The sensor(s), for example, is configured to detect impedance of the first electrode pad 1118 and the second electrode pad 1130. In some cases, the sensor(s) include one or more one or more accelerometers, one or more humidity sensors, and one or more temperature sensors. The sensor(s), for instance, is configured to detect one or more conditions to which the electrodes have been exposed. According to some implementations, the signals transmitted by the transceiver(s) 1110 indicate the t state parameter(s) of the accessory devices.

The device 1102 further includes at least one output device 1108, in various implementations. Examples of the output device(s) 1108 include, for instance, at least one of a display (e.g., a projector, an LED screen, etc.), a speaker, a haptic output device, a printer, a light such as an LED, or any combination thereof. In some implementations, the output device(s) 1108 include a screen configured to display various parameters detected by and/or reported to the device 1102.

The device 1102 further includes memory/storage component 1104 stored in computer readable media 1124. The computer readable media 1124 is illustrated as including memory/storage component 1104. The memory/storage component 1104 represents memory/storage capacity associated with one or more computer-readable media. The memory/storage component 1104 may include volatile media (such as random access memory (RAM)) and/or nonvolatile media (such as read-only memory (ROM), Flash memory, optical disks, magnetic disks, and so forth). The memory/storage component 812 may include fixed media (e.g., RAM, ROM, a fixed hard drive, and so on) as well as removable media (e.g., Flash memory, a removable hard drive, an optical disc, and so forth). The computer-readable media 1124 may be configured in a variety of other ways as further described below.

The memory/storage component 1104 stores instructions that, when executed by the processor(s) 1112, causes the processor(s) 1112 to perform various operations. In various examples, the memory/storage component 1104 stores methods, threads, processes, applications, objects, modules, any other sort of executable instruction, or a combination thereof. In some cases, the memory/storage component 1104 stores files, databases, or a combination thereof. In various cases, the memory/storage component 1104 stores instructions, programs, threads, objects, data, or any combination thereof, that cause the processor(s) 1112 to perform various functions. In various cases, the memory/storage component 1104 stores one or more parameters that are detected by the device 1102 and/or reported to the device 1102.

In implementations of the present disclosure, the memory/storage component 1104 also stores one or more components including feature selector 1128, predictive model 1120, and report generator 1132. The component(s) include programs, instructions, files, databases, models, or any other type of data that causes the device 1102 to perform any of the functions described herein.

### Example Clauses

The Example Clauses and Example(s) below are included to demonstrate particular implementations of the disclosure. Those of ordinary skill in the art should recognize in light of the present disclosure that many changes can be made to the specific implementations disclosed herein and still obtain a like or similar result without departing from the spirit and scope of the disclosure.

1. A system, including an accessory device for a defibrillator, the accessory device including an electrode, an electrode gel including a dielectric material disposed on a surface of the electrode and a sensor configured to detect an environmental condition of the accessory device; a computing device including: a processor configured to: determine an impedance of the electrode gel; and predict a lifespan of the accessory device using the impedance of the electrode gel and the environmental condition of the accessory device; and an output device configured to output the lifespan of the accessory device.

2. The system of clause 1, wherein the sensor includes a humidity sensor, a temperature sensor, or a motion sensor.

3. The system of clause 1 or 2, wherein the processor is further configured to determine a signal-to-noise ratio of the electrode, and wherein the processor is configured to predict the lifespan of the accessory device further using the signal-to-noise ratio of the electrode.

4. A testing device, including: an accessory device including: an electrode; an electrode gel disposed on a surface of the electrode; and a sensor configured to detect an impedance of the electrode gel; an output device; and a processor configured to: identify a trend in the impedance of the electrode gel; predict, by analyzing the trend, an estimated life remaining of the accessory device; and cause the output device to output an indication of the estimated life remaining of the accessory device on the output device.

5. The testing device of clause 4, wherein the processor is further configured to: determine a signal-to-noise ratio of the electrode, and wherein the processor is configured to predict the estimated life remaining of the electrode further by analyzing the signal-to-noise ratio.

6. The testing device of clause 5, wherein the processor is configured to predict the estimated remaining life of the accessory device by: detecting a change in the signal-to-noise ratio over time that is greater than a threshold change; and determining that the estimated life remaining in the accessory device is below a threshold time period.

7. The testing device of any of clauses 4 to 6, wherein the accessory device further includes an environmental sensor configured to detect an environmental condition of the accessory device, and wherein the processor is further configured to predict the estimated life remaining of the accessory device further by analyzing the environmental condition of the testing device.

8. The testing device of clause 7, wherein the environmental sensor includes a temperature sensor or a humidity sensor.

9. The testing device of any of clauses 4 to 8, wherein the processor is further configured to: identify a number of times the accessory device has been used, and wherein the processor is configured to predict the estimated life remaining of the accessory device further by analyzing the number of times the accessory device has been used.

10. The testing device of any of clauses 4 to 9, wherein the accessory device further includes a motion sensor configured to detect a motion of the accessory device, and wherein the processor is configured to predict the estimated life remaining of the accessory device further by analyzing the motion of the accessory device.

11. The testing device of any of clauses 4 to 10, wherein the accessory device further includes a battery, and wherein the processor is further configured to: determine a charging rate of the battery of the accessory device; and wherein the processor is configured to predict the estimated life remaining of the accessory device further by analyzing the charging rate.

12. The testing device of any of clauses 4 to 11, wherein the processor is further configured to: determine an electrical resistance of the electrode; and wherein the processor is configured to predict the estimated life remaining of the accessory device further by analyzing the resistance of the electrode.

13. The testing device of any of clauses 4 to 12, wherein the testing device is stored within a medical device.

14. The testing device of clause 13, wherein the medical device is a defibrillator.

15. A method including: measuring, via an impedance sensor, an electrical impedance of an electrode gel of an accessory device; determining a trend of the electrical impedance of the electrode gel over time; determining a life expectancy of the accessory device by inputting the trend of the electrical impedance of the electrode gel over time into a trained predictive model, the predictive model being trained by training data including previous measurements of electrical impedances of other electrode gels; and outputting the life expectancy of the accessory device.

16. The method of clause 15, further including: detecting an environmental condition of the accessory device, the environmental condition including a temperature or a humidity, wherein determining the life expectancy of the accessory device further includes inputting the environmental condition into the trained predictive model.

17. The method of clause 15 or 16, further including: detecting a number of times the accessory device has been used, wherein determining the life expectancy of the accessory device further includes inputting the number of times the accessory device has been used into the trained predictive model.

18. The method of any of clauses 15 to 17, further including: detecting a signal-to-noise ratio of the accessory device, wherein determining the life expectancy of the accessory device further includes inputting the signal-to-noise ratio of the accessory device into the trained predictive model.

19. The method of any of clauses 15 to 18, further including: detecting a charging rate of a battery of the accessory device, wherein determining the life expectancy of the accessory device further includes inputting the charging rate of the battery of the accessory device into the trained predictive model.

20. The method of any of clauses 15 to 19, wherein the life expectancy of the accessory device includes an expected expiration date of the accessory device.

21. A testing device including: an input device configured to determine an impedance of a gel disposed on an electrode of an accessory device; and a processor configured to: predict, by analyzing the impedance of the gel, a lifespan of the accessory device; and issue an alert when the lifespan of the accessory device is lower than a threshold lifespan.

22. The testing device of clause 21, wherein the input device includes: a sensor configured to detect the impedance of the gel disposed on the electrode of the accessory device; or a receiver configured to receive a communication signal indicating the impedance of the gel disposed on the electrode of the accessory device.

23. The testing device of clause 21 or 22, wherein the processor is further configured to: identify an expected geographic location of the accessory device, and wherein the processor is configured to predict the lifespan of the accessory device by further analyzing the expected geographic location of the accessory device.

24. The testing device of any of clauses 21 to 23, further including: a temperature sensor configured to take measurements of a temperature of the accessory device, and wherein the processor is configured to predict the lifespan of the accessory device further by analyzing the measurements of the temperature of the accessory device.

25. The testing device of any of any of clauses 21 to 24, further including: a humidity sensor configured to take measurements of a humidity of the accessory device, wherein the processor is configured to predict the lifespan of the accessory device further by analyzing the measurements of the humidity of the accessory device.

26. An accessory device, including: a plug configured to be coupled to an external defibrillator; an electrode; a gel coated on the electrode and including a dielectric material; a conductive trace embedded in the plug; a sensor electrically coupled to the electrode and the conductive trace, the sensor being configured to detect an impedance of the gel; an output device configured to output an alert indicating that a lifespan of the electrode is below a threshold level; an electrically insulative substrate disposed on the electrode, the electrode being disposed between the electrically insulative substrate and the gel; and a housing enclosing the plug, the electrode, the electrically insulative substrate, and the gel.

27. The accessory device of clause 26, further including: a temperature sensor enclosed by the housing, the temperature sensor being configured to detect that a temperature within the housing exceeds a threshold temperature, and wherein the output device is configured to output the alert in response to the temperature sensor detecting that the temperature within the housing exceeds the threshold temperature.

28. The accessory device of clause 26 or 27, further including: a humidity sensor enclosed by the housing, the humidity sensor being configured to detect that a humidity within the housing exceeds a threshold humidity, and wherein the output device is configured to output the alert in response to the humidity sensor detecting that the humidity within the housing exceeds the threshold humidity.

29. The accessory device of any of clauses 26 to 28, further including: a motion sensor configured to detect that an acceleration of the accessory device has exceeded a threshold acceleration, wherein the output device is configured to output the alert in response to the motion sensor detecting that the acceleration of the accessory device has exceeded the threshold acceleration.

30. The accessory device of any of clauses 26 to 29, wherein the output device includes a near field communication (NFC) antenna.

31. A method including: detecting an impedance of a gel coated on an electrode, the gel including a dielectric material; determining a trend of the impedance over time; determining a lifespan of the electrode based on the trend in the impedance of the gel; determining that the lifespan of the electrode is below a threshold; and in response to determining that the lifespan of the electrode is below the threshold, outputting an alert.

32. The method of clause 31, further including: detecting an environmental condition of a gel environment, the environmental condition including a temperature or a humidity, wherein determining the lifespan of the electrode further includes determining an average environmental condition of the gel environment.

33. The method of clause 32, further including: detecting motion of the gel environment, wherein determining the lifespan of the electrode further includes determining that motion of the gel environment has been above a threshold amount.

34. The method of any of clauses 31 to 33, further including: detecting a geographic location of the electrode, wherein determining the lifespan of the electrode includes analyzing the geographic location.

35. The method of any of clauses 31 to 34, further including: detecting a signal-to-noise ratio of the electrode; determining a trend in the signal-to-noise ratio over time, wherein determining the lifespan of the electrode further includes analyzing the trend in the signal-to-noise ratio over time.

36. The method of any of clauses 31 to 35, further including: determining an electrical resistance of the electrode; and determining a trend of the electrical resistance over time, wherein, determining a lifespan of the electrode further includes determining that the electrical resistance of the electrode is above a threshold.

37. The method of any of clauses 31 to 36, further including outputting the alert to a display when the lifespan is below the threshold.

38. The method of any of clauses 31 to 37, further including outputting an audible signal when the lifespan of the electrode is below the threshold.

39. The method of any of clauses 31 to 38, further including outputting a visible physical signal when the lifespan is below the threshold.

40. The method of clause 39, wherein the physical signal is a change in color.

41. The method of clause 39, wherein the physical signal is a pop-up button.

42. The method of clause 39, wherein the physical signal is a change in a light.

43. The method of clause 39, wherein the visible physical signal is graded.

44. The method of any of clauses 31-43, wherein detecting the impedance of a gel comprises a closed loop test.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain implementations are described herein, including the best mode known to the inventors for carrying out implementations of the disclosure. Of course, variations on these described implementations will become apparent to those of ordinary skill in the art upon reading the foregoing description. The inventors expect skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

What is claimed is:

1. A system, comprising:
an accessory device for a defibrillator, the accessory device comprising:
an electrode;
an electrode gel comprising a dielectric material disposed on a surface of the electrode; and
a sensor configured to detect an environmental condition of the accessory device;
a computing device comprising:
a processor configured to:
determine an impedance of the electrode gel; and
predict a lifespan of the accessory device using the impedance of the electrode gel and the environmental condition of the accessory device; and
an output device configured to output the lifespan of the accessory device.

2. The system of claim 1, wherein the sensor comprises a humidity sensor, a temperature sensor, or a motion sensor.

3. The system of claim 1, wherein the processor is further configured to determine a signal-to-noise ratio of the electrode, and wherein the processor is configured to predict the lifespan of the accessory device further using the signal-to-noise ratio of the electrode.

4. A testing device, comprising:
an accessory device comprising:
an electrode;
an electrode gel disposed on a surface of the electrode; and
a sensor configured to detect an impedance of the electrode gel;
an output device; and
a processor configured to:
identify a trend in the impedance of the electrode gel;
predict, by analyzing the trend, an estimated life remaining of the accessory device; and
cause the output device to output an indication of the estimated life remaining of the accessory device on the output device.

5. The testing device of claim 4, wherein the processor is further configured to:
determine a signal-to-noise ratio of the electrode, and wherein the processor is configured to predict the estimated life remaining of the electrode further by analyzing the signal-to-noise ratio.

6. The testing device of claim 5, wherein the processor is configured to predict the estimated remaining life of the accessory device by:
detecting a change in the signal-to-noise ratio over time that is greater than a threshold change; and
determining that the estimated life remaining in the accessory device is below a threshold time period.

7. The testing device of claim 4, wherein the accessory device further comprises an environmental sensor configured to detect an environmental condition of the accessory device, and wherein the processor is further configured to predict the estimated life remaining of the accessory device further by analyzing the environmental condition of the testing device.

8. The testing device of claim 7, wherein the environmental sensor comprises a temperature sensor or a humidity sensor.

9. The testing device of claim 4, wherein the processor is further configured to:
identify a number of times the accessory device has been used, and wherein the processor is configured to predict the estimated life remaining of the accessory device further by analyzing the number of times the accessory device has been used.

10. The testing device of claim 4, wherein the accessory device further comprises a motion sensor configured to detect a motion of the accessory device, and wherein the processor is configured to predict the estimated life remaining of the accessory device further by analyzing the motion of the accessory device.

11. The testing device of claim 4, wherein the accessory device further comprises a battery, and wherein the processor is further configured to:
determine a charging rate of the battery of the accessory device; and wherein the processor is configured to predict the estimated life remaining of the accessory device further by analyzing the charging rate.

12. The testing device of claim 4, wherein the processor is further configured to:
determine an electrical resistance of the electrode; and wherein the processor is configured to predict the estimated life remaining of the accessory device further by analyzing the resistance of the electrode.

13. The testing device of claim 4, wherein the testing device is stored within a medical device.

14. The testing device of claim 13, wherein the medical device is a defibrillator.

15. A method comprising:
measuring, via an impedance sensor, an electrical impedance of an electrode gel of an accessory device;
determining a trend of the electrical impedance of the electrode gel over time;
determining a life expectancy of the accessory device by inputting the trend of the electrical impedance of the electrode gel over time into a trained predictive model, the predictive model being trained by training data comprising previous measurements of electrical impedances of other electrode gels; and
outputting the life expectancy of the accessory device.

16. The method of claim 15, further comprising:
detecting an environmental condition of the accessory device, the environmental condition comprising a temperature or a humidity,
wherein determining the life expectancy of the accessory device further comprises inputting the environmental condition into the trained predictive model.

17. The method of claim 15, further comprising:
detecting a number of times the accessory device has been used,
wherein determining the life expectancy of the accessory device further comprises inputting the number of times the accessory device has been used into the trained predictive model.

18. The method of claim 15, further comprising:
detecting a signal-to-noise ratio of the accessory device,
wherein determining the life expectancy of the accessory device further comprises inputting the signal-to-noise ratio of the accessory device into the trained predictive model.

19. The method of claim 15, further comprising:
detecting a charging rate of a battery of the accessory device,
wherein determining the life expectancy of the accessory device further comprises inputting the charging rate of the battery of the accessory device into the trained predictive model.

20. The method of claim 15, wherein the life expectancy of the accessory device comprises an expected expiration date of the accessory device.
